(19) United States
(12) Patent Application Publication
(10) Pub. No.: US 2010/0196328 A1
(43) Pub. Date: Aug. 5, 2010

(54) ISCHEMIA-INDUCED NEOVASCULARIZATION IS ENHANCED BY hCNS-SC TRANSPLANTATION

(76) Inventors: Tonya Bliss, Menlo Park, CA (US); Gary K. Steinberg, Redwood City, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/596,884

(22) PCT Filed: Apr. 24, 2008
(86) PCT No.: PCT/US08/61390

§ 371 Date: Apr. 14, 2010

Related U.S. Application Data
(60) Provisional application No. 60/926,429, filed on Apr. 25, 2007.

(51) Int. Cl. A61K 35/30; A61P 25/00

(52) U.S. Cl. 424/93.7

(57) ABSTRACT

The invention provides methods for inducing or enhancing neovascularization following ischemia by transplanting an effective amount of human central nervous system stem cells. The human central nervous system stem cells can be grown as neurospheres or in adherent culture. Also provided are methods for inducing the repair of ischemic tissue in a patient and methods for treating stroke in a patient suffering therefrom.

### RELATED APPLICATIONS

This application claims the benefit of U.S. Ser. No. 60/926,429, filed Apr. 25, 2007, the contents of which is incorporated herein by reference in its entirety:

### GRANT SUPPORT

This invention was made with United States Government support under Contract No. NS37520 by the National Institutes of Health. The United States Government has certain rights in the invention.

### FIELD OF THE INVENTION

This invention relates generally to methods of neurotransplantation of multipotent neural stem cells to induce neovascularization.

### BACKGROUND OF THE INVENTION

Blood vessels are damaged or lost during ischemia. It has previously been observed that, following ischemia, neovascularization (e.g., angiogenesis and/or vasculogenesis) occurs in the penumbra. Moreover, the extent of new blood vessel formation is associated with neurological recovery. (See, e.g., Senior, Lancet 358:817 (2001); Krupinski, Lancet 342:742 (1993)). At present there is no proven therapy for stroke, except thrombolytic treatments which have limited efficacy and must be administered within the first few hours after stroke.

Thus, development of a cell-based therapy for repair of ischemic tissue and/or treatment of stroke is desired.

### SUMMARY OF THE INVENTION

Provided herein are methods of inducing or enhancing neovascularization following ischemia in a patient suffering therefrom by transplanting an effective amount of human central nervous system stem cells (hCNS-SC) to one or more lesioned regions of the central nervous system of the patient. The hCNS-SC to be transplanted can be grown as neurospheres (hCNS-SCns) or in adherent culture. For example, the lesioned region of the patient's central nervous system is the ischemic cortex. According to these methods, the transplantation of the hCNS-SCs results in increased angiogenesis, increased vasculogenesis, or both within the cortical ischemic penumbra. In various embodiments, at least 1×10⁵ cells are injected at each lesioned region.

Also provided are methods for inducing the repair of ischemic tissue in a patient, the method by transplanting an effective amount of hCNS-SC at one or more loci within the central nervous system of the patient, wherein the hCNS-SC are able to increase angiogenesis, vasculogenesis, or both within the ischemic tissue. For example, the hCNS-SC can be grown as neurospheres (hCNS-SCns) or in adherent culture, and at least 1×10⁵ cells are injected per loci.

The invention also describes a method for treating a stroke in a patient by injecting at least 1×10⁵ human central nervous system stem cells (hCNS-SC) at one or more loci within the ischemic cortex of the central nervous system of the patient suffering therefrom. The injected hCNS-SC are able to increase neovascularization in the ischemic cortex, thereby aiding neurological recovery in the stroke patient. Those skilled in the art will recognize that the hCNS-SC can be grown as neurospheres (hCNS-SCns) or in adherent culture.

In any of the embodiments described herein, the hCNS-SCs can be obtained from the host's own neural tissue. hCNS-SCs can be obtained, for example from cerebral cortex tissue, cerebellum tissue, midbrain tissue, brainstem tissue, spinal cord tissue, ventricular tissue, frontal lobe tissue, conus medularis tissue, hypothalamus tissue, or a combination thereof. Moreover, the human neural stem cells to be transplanted can be obtained from embryonic, fetal, juvenile, or adult human neural tissue.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a series of images and graphs showing that neovascularization following ischemia is enhanced following hCNS-SCns transplantation. FIG. 1A is a schematic representation of regions of interest (“ROIs”) imaged in the dorsal ischemic penumbra. FIG. 1B is a series of representative images showing (i) blood vessel staining in the contralateral cortex (results shown are similar at 1 week, 5 weeks, and 16 weeks following distal middle cerebral artery occlusion (“dMCAo”)); (ii) blood vessel staining in the ipsilateral cortex at 1 week post-dMCAo; (iii) blood vessel staining in the ipsilateral cortex at 5 weeks post-dMCAo; and (iv) blood vessel staining in the ipsilateral cortex at 5 weeks post-dMCAo for rats given human central nervous system stem cells (“hCNS-SCns”). The “*” in FIG. 1B indicates the edge of the lesion. FIG. 1C is a graph showing the quantification of blood vessel density (“BVD”). In this graph, n=4-6 for the dMCAo+ cells and dMCAo+ buffer groups and n=2 for all other groups.

FIG. 2 is a graph showing nude rat blood vessel density staining at 7 weeks post-transplantation. Even though the nude rats lack T cells, enhanced neovascularization is observed in the cell-transplanted group. This finding suggests that T-cells are not important for the observed effect of hCNS-SCns on neovascularization.

FIG. 3 is a series of photomicrographs demonstrating that most of the new blood vessels that are formed following hCNS-SCns transplantation are functional (i.e., they are perfused). As shown, these newly formed blood vessels stain for both b-DG (which stains A blood vessels) and lectin (which stains only those vessels that receive blood). A small number of the new blood vessels were possibly not functional in the penumbra (see arrows). However, no non-functional blood vessels were observed in the contralateral cortex.

FIG. 4 is an image and a graph showing neovascularization in the ventral cortex is not affected by hCNS-SCns transplantation in the dorsal cortex. FIG. 4A is a schematic showing the lesion site (grey area) and ROIs imaged in the ischemic penumbra. FIG. 4B is a graph showing quantification of ventral BVD in the same animals and brain slices as used in FIG. 1.

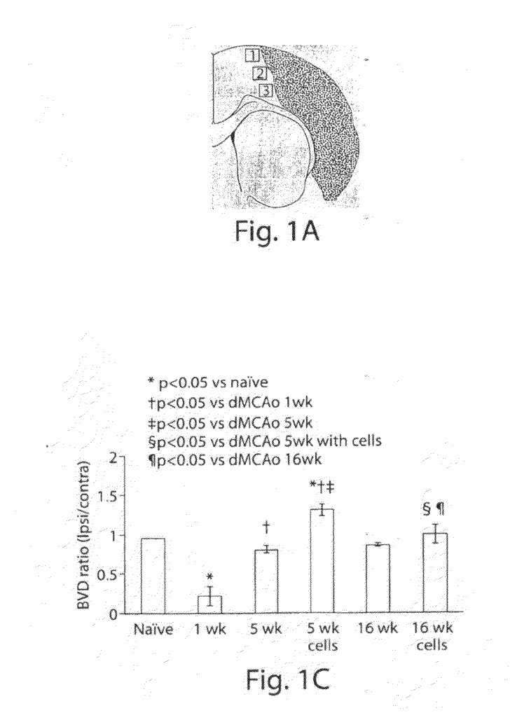

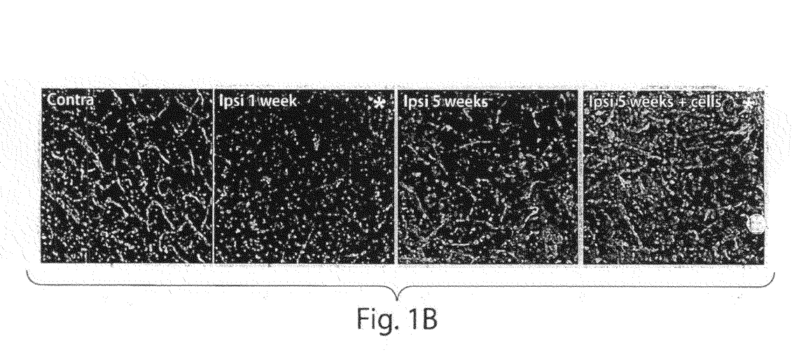

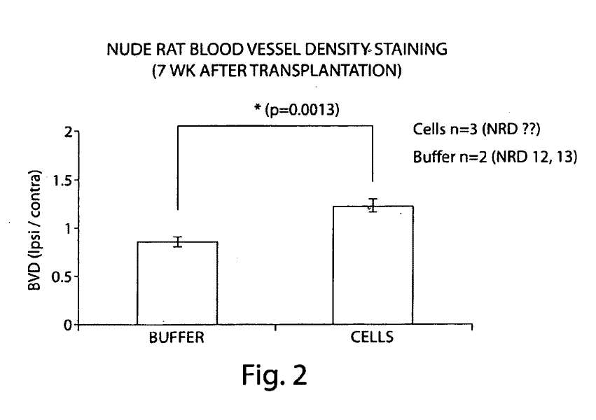

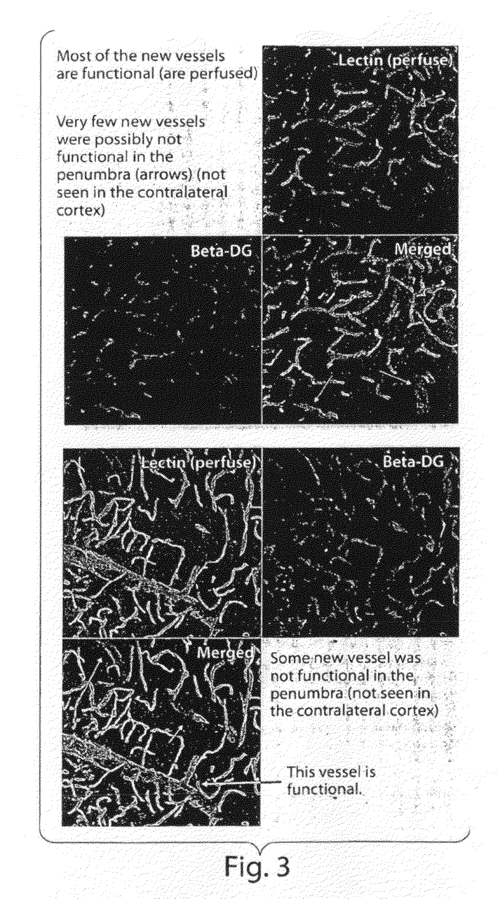

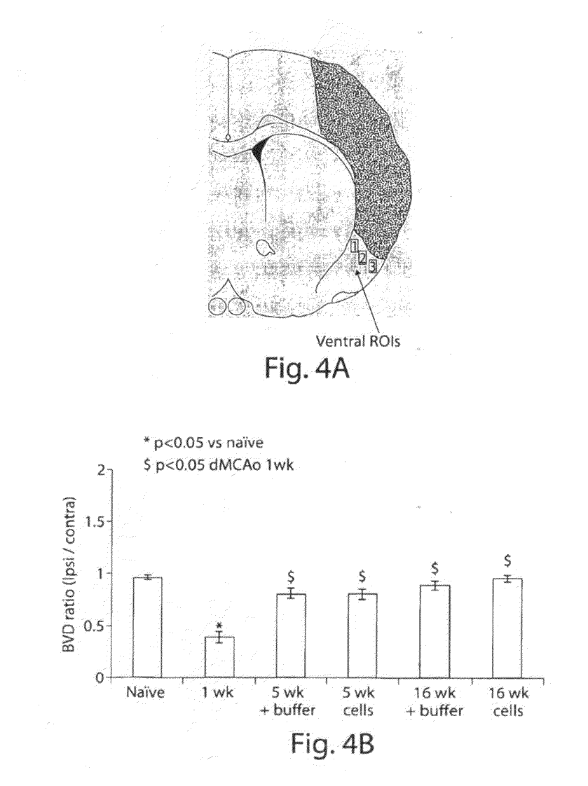

### DETAILED DESCRIPTION

### Central Nervous System Stem Cells

During development of the central nervous system (“CNS”), multipotent precursor cells (also known as neural stem cells) proliferate and give rise to transiently dividing progenitor cells that eventually differentiate into the cell types that compose the adult brain. Neural stem cells are classically defined as having the ability to self-renew (i.e., form more stem cells), to proliferate, and to differentiate into multiple different phenotypic lineages, including neurons, astrocytes and oligodendrocytes.

The non-stem cell progeny of neural stem cells are typically referred to as “progenitor cells”. Progenitor cells are capable of giving rise to various cell types within one or more lineages. Thus, the term “neural progenitor cell” refers to an undifferentiated cell derived from a neural stem cell. It is not itself a stem cell. Some progenitor cells can produce progeny that are capable of differentiating into more than one cell type. A distinguishing feature of a progenitor cell is that, unlike a stem cell, it does not exhibit self maintenance, and it typically is thought to be committed to a particular path of differentiation and it will, under appropriate conditions, eventually differentiate into glia or neurons.

The term “precursor cells” refers to the progeny of neural stem cells, and, thus, includes both progenitor cells and daughter neural stem cells.

Neural stem cells have been isolated from several mammalian species, including mice, rats, pigs and humans. (See, e.g., WO 93/01275; WO 94/09119; WO 94/10292; WO 94/16718; U.S. Pat. No. 5,968,829; and Cattaneo et al., Mol. Brain Res., 42, pp. 161-66 (1996), all herein incorporated by reference). Multipotent neural stem cells can be obtained from embryonic, post-natal, juvenile, or adult neural tissue. For example, neural stem cells can be obtained from the cerebral cortex, cerebellum, midbrain, brainstem, spinal cord, and ventricular tissue, as well as areas of the PNS including the carotid body and the adrenal medulla. Other preferred areas include regions in the basal ganglia, preferably the striatum, which consists of the caudate and putamen, or various cell groups such as the globus pallidus, the subthalamic nucleus, the nucleus basalis, the substantia nigra pars compacta, as well as from ventricular tissue found lining CNS ventricles, including the subependyma.

A population of cells exists within the adult CNS that exhibit stem cell properties in their ability to self-renew and to produce the differentiated mature cell phenotypes of the adult CNS. These stem cells are found throughout the CNS and particularly in the subventricular region and the dentate gyrus of the hippocampus. Neural stem cells have also been isolated from a variety of adult CNS ventricular regions, including the frontal lobe, conus medullaris, thoracic spinal cord, brain stem, and hypothalamus.

Growth factor-responsive stem cells can be isolated from many regions of the neuraxis and at different stages of development, of murine, rodent, mammalian, and human CNS tissue. These cells vary in their response to growth factors such as EGF, basic FGF (bFGF, FGF-2) and transforming growth factor alpha (TGFα) and can be maintained and expanded in culture in an undifferentiated state for long periods of time. (See, e.g. WO93/01275 and WO94/16788, incorporated herein by reference).

Neural stem cells can also be cultured in a culture medium containing leukemia inhibitory factor (“LIF”), which markedly and unexpectedly increases the rate of proliferation of neural stem cells, particularly human neural stem cells. For example, the medium may contain cell viability and cell proliferation effective amounts of the following components:
(a) a standard culture medium being serum-free (containing 0-0.49% serum) or serum-depleted (containing 0.5-5.0% serum), known as a “defined” culture medium, such as Iscove's modified Dulbecco's medium (“IMDM”), RPMI, DMEM, Fischer's, alpha medium, Leibovitz's, L-15, NCTC, F-10, F-12, MEM and McCoy's;
(b) a suitable carbohydrate source, such as glucose;
(c) a buffer such as MOPS, HEPES or Tris, preferably HEPES;
(d) a source of hormones including insulin, transferrin, progesterone, selenium, and putrescine;
(e) one or more growth factors that stimulate proliferation of neural stem cells, such as EGF, bFGF, PDGF, NGF, and analogs, derivatives and/or combinations thereof, preferably EGF and bFGF in combination;
(f) LIF

The identification, culture, growth, and use of mammalian, including human, neural stem cell cultures, either as suspension cultures or as adherent cultures, is disclosed in Weiss et al., U.S. Pat. No. 5,750,376 and Weiss et al., U.S. Pat. No. 5,851,832, both incorporated herein by reference. Similarly, Johe, U.S. Pat. No. 5,753,506, incorporated herein by reference, refers to the proliferation of CNS neural stem cells in adherent cultures. By way of non-limiting example, CNS neural stem cells can be plated at a density of 1×10⁶ cells (from hippocampus and septum) or 1.5×10⁶ cells (from other CNS regions) per 10 cm plate and expanded in serum-free culture medium containing 10 ng/ml of a mitogenic growth factor (e.g., bFGF and/or EGF).

When cultured in suspension, CNS neural stem cell cultures typically form neurospheres (e.g., a cluster of undifferentiated cells). After about 4 to 5 days in the absence of a substrate, the proliferating neurospheres were off the floor of the culture dish and tend to form the free-floating clusters characteristic of neurospheres. The proliferating precursor cells of the neurosphere continue to proliferate in suspension. After about 3-10 days in vitro, and more particularly after about 6-7 days in vitro, the proliferating neurospheres are fed every 2-7 days, preferably every 2-4 days by gentle centrifugation and resuspension in a culture medium containing a growth factor.

The neurospheres of the suspension culture can be easily passaged to reinitiate proliferation. For example, the cells within the neurosphere can be dissociated via mechanical trituration, trypsinization, treatment with collagenase, or the like. (See, e.g., U.S. Pat. Nos. 6,238,922 and 7,049,141, which are herein incorporated by reference). Single cells dissociated from the neurospheres can be suspended in a cell culture medium containing a growth factor. A percentage of these cells will proliferate and form new neurospheres largely composed of undifferentiated cells. This procedure can be continued until the desired number of precursor cells is obtained.

The cells of a single neurosphere or adherent culture are clonal in nature because they are the progeny of a single neural stem cell. In the continued presence of a proliferation-inducing growth factor such as EGF or the like, precursor cells within the neurosphere continue to divide, thereby resulting in an increase in the size of the neurosphere and the number of undifferentiated neural cells. Neurospheres are not immunoreactive for neurofilament (NF; a marker for neurons), neuron-specific enolase (NSE; a marker for neurons) or myelin basic protein (MBP; a marker for oligodendrocytes). However, the cells within the neurosphere are immunoreactive for nestin, an intermediate filament protein found in many types of undifferentiated CNS cells. (See Lehndahl et al., Cell 60:585-595 (1990), incorporated herein by reference). Antibodies are available to identify nestin, including, for example, the rat antibody referred to as Rat401. If neurospheres are cultured under conditions that allow differentiation, progenitor cells differentiate to neurons and glia. These mature phenotypes associated with the differentiated cell types that may be derived from the neural stem cell progeny are predominantly negative for the nestin phenotype.

Human central nervous system stem cell-derived neurospheres (“HuCNS-SC™”) (also referred to herein as “human central nervous system stem cells” or “hCNS-SC”, or “human central nervous system stem cells grown as neurospheres” or “hCNS-SCns”) (StemCells, Inc., Palo Alto, Calif.) are a somatic cell therapy product comprised of a homogeneous aseptic suspension of neural progenitor cells capable of migrating from the implantation site and differentiating into mature cell types of the brain.

The human neural stem cells described herein can be genetically engineered according to known methodology. A gene of interest (i.e., a gene that encodes a biologically active molecule) can be inserted into a cloning site of suitable expression vector by using standard techniques. These techniques are well known to those skilled in the art. (See, e.g., WO94/16718, incorporated herein by reference).

The expression vector containing the gene of interest may then be used to transfect the desired cell line. Standard transfection techniques such as calcium phosphate co-precipitation, DEAE-dextran transfection, electroporation, biolistics, or viral transfection may be utilized. Commercially available mammalian transfection kits may be purchased from i.e., Stratagene. Human adenoviral transfection may be accomplished as described in Berg et al. Exp. Cell Res., 192, pp. (1991). Similarly, lipofectamine-based transfection May be accomplished as described in Cattaneo, Mol. Brain Res., 42, pp. 161-66 (1996).

A wide variety of host/expression vector combinations may be used to express a gene encoding a biologically active molecule of interest. See, e.g., U.S. Pat. No. 5,545,723, herein incorporated by reference, for suitable cell-based production expression vectors.

Increased expression of the biologically active molecule can be achieved by increasing or amplifying the transgene copy number using amplification methods well known in the art. Such amplification methods include, e.g., DHFR amplification (see, e.g., Kaufman et al., U.S. Pat. No. 4,470,461) or glutamine synthetase (“GS”) amplification (see, e.g., U.S. Pat. No. 5,122,464, and European published application EP 338,841, all herein incorporated by reference).

The neural stem cells described herein, and their differentiated progeny may be immortalized or conditionally immortalized using known techniques. Preferably, the stem cells and/or their differentiated progeny are conditionally immortalized. Among the conditional immortalization techniques contemplated are Tet-conditional immortalization (see WO96/31242, incorporated herein by reference), and Mx-1 conditional immortalization (see WO96/02646, incorporated herein by reference).

### Transplantation of Central Nervous System Stem Cells

It is well recognized in the art that transplantation of tissue into the CNS offers the potential for treatment of neurodegenerative disorders and CNS damage due to injury. (See, e.g., Lindvall, TINS 14 (8):376-383 (1991)). Moreover, as described herein, transplantation of hCNS-SC also offers the potential for inducing neovascularization following ischemia.

Transplantation of new cells into the CNS has the potential to repair damaged circuitries and to provide deficient, defect, or missing biologically active molecules, thereby restoring function. However, the absence of suitable cells for transplantation purposes has prevented the full potential of this procedure from being met. “Suitable” cells are cells that meet the following criteria: 1) cells that can be obtained in large numbers; 2) cells that can be proliferated in vitro to allow insertion of genetic material, if necessary; 3) cells that are capable of surviving indefinitely but stop growing after transplantation to the brain; 4) cells that are non-immunogenic and are preferably obtained from a patient's own tissue; and 5) cells that are able to form normal neural connections and respond to neural physiological signals. (See Bjorklund TINS 14 (8):319-322 (1991). The progeny of multipotent neural stem cells obtainable from embryonic, juvenile, or adult mammalian CNS tissue, which are able to divide indefinitely when maintained in vitro meet all of the desirable requirements of cells suitable for neural transplantation purposes and are a particularly suitable cell line as the cells have not been immortalized and are not of tumorigenic origin.

hCNS-SC can also be administered to patients to increase neovascularization following ischemia.

In some instances, it may be possible to prepare hCNS-SC from the recipient's own nervous system (e.g., in the case of tumor removal biopsies etc.). In such instances, the neural stem cell progeny may be generated from dissociated tissue and proliferated in vitro using any suitable method known to those of ordinary skill in the art. Upon suitable expansion of cell numbers, the hCNS-SC cells may be harvested, genetically modified if necessary, and readied for direct injection into the recipient's CNS.

For transplants into human patients, those skilled in the art will recognize that any suitable method for the transplantation, administration, injection, and/or implantation of hCNS-SC can be employed in patients. (See, e.g., U.S. Pat. No. 6,497,872, incorporated herein by reference). For example, an effective amount of cells can be injected directly into the ischemic cortex. The determination of an effective amount of hCNS-SC to be transplanted is within the level of ordinary skill in the art. For example, the total number of cells to be transplanted can be up 1×10⁹, e.g., up to 1×10⁸, up to 1×10⁷, up to 1×10⁶, up to 1×10⁵, or up to 1×10⁴ cells.

In accordance with the methods described herein, hCNS-SC can be injected into multiple (e.g., 2, 3, 4, 5, 6 or more) loci within the ischemic cortex. In some embodiments, 100,000 cells can be transplanted at 3 sites, for a total of 300,000 cells. In other embodiments, a total of up to 1.5×10⁶ cells can be injected (at one or more sites). Additionally, by way of nonlimiting example, in yet another embodiment, in humans, 1 ml of cells (at a maximum cell concentration of 1×10⁸/ml) cells can be transplanted into 2-4 (or more) sites. In this embodiment, the maximum cell dose to be transplanted would be approximately 2-4×10⁸ (or more) cells. Those skilled in the art will recognize that the total number of cells that are transplanted can be increased by increased the number of injection sites.

Transplantation of hCNS-SCs to Enhance Neovascularization

As used herein, the term “neovascularization” is defined as a process of tissue vascularization involving the growth of new and/or developing blood vessels in a tissue. It is mediated by the proliferation or infiltration of endothelial cells and smooth muscle cells. Those skilled in the art will recognize that neovascularization can proceed in one of three ways: (i) new vessels can sprout from preexisting ones (“angiogenesis”); (ii) de novo development of vessels can arise from mobilization and infiltration of endogenous endothelial precursor cells (“vasculogenesis”); and/or (iii) existing small vessels can enlarge in diameter.

In order to examine the effects of hCNS-SCns on neurovascularization, male Sprague Dawley rats were subjected to the distal middle cerebral artery occlusion (“dMCAo”) model of stroke. Six days later, the rats were started on an immunosuppression regime of daily intraperitoneal injections of cyclosporine A (10 mg/kg) which was continued until the animals were sacrificed. On day 7 post-stroke, fetal human neurospheres derived from CNS stem cells grown as neurospheres (“hCNS-SCns”) were transplanted into three sites the ischemic cortex (1×10⁵ cells per site in 1 μl). Another set of stroke rats, which were also given cyclosporine A and served as the control group, received buffer injections at the same cortical coordinates.

The animals were sacrificed at 1, 5 and 16 weeks post-dMCAo and brain sections stained with the blood vessel marker beta-dystroglycan. Fluorescent images were taken in three ROIs in both the dorsal and ventral cortical ischemic penumbra and in the equivalent location in the contralateral cortex.

Blood vessel density (“BVD”) was measured by Image J. The extent of neovascularization was determined by measuring the blood vessel density (BVD) in the ROIs in the ischemic penumbra and normalizing using the BVD in the equivalent regions in the contralateral hemisphere. There were no significant changes in the contralateral BVDs after stroke or transplantation (as determined by one way ANOVA), therefore all changes in the BVD ratio were a result of changes in the ipsilateral neovascularization. The results obtained demonstrate that both dorsal and ventral ipsilateral BVD is significantly reduced compared to naive (or sham) controls in the penumbra at 1 week post stroke. However, BVD is significantly improved by 5 weeks post stroke (p<0.05). In the ventral penumbra, which is far from the region of hCNS-SC engraftment (see Kelly et al., PNAS 101:11839 (2004)), there was no difference in BVD in vehicle- and hCNS-SC-treated animals. However, in the dorsal penumbra, which is in the vicinity of hCNS-SC-treated animals, rats receiving hCNS-SCns grafts showed enhanced neovascularization at 5 weeks post-stroke as compared to vehicle-treated rats (p<0.05).

Although ipsilateral BVD is higher than contralateral BVD in the cell-grafted animals, the caliber of the blood vessels is different. Specifically, the vessels found in the ipsilateral side appear shorter and less branched. At 16 weeks post-stroke, the ipsilateral BVD was still significantly higher in cell-grafted animals (p<0.05), but it was significantly reduced compared to that measured at 5 weeks (p<0.05), implying that the new vessels are transient.

This data demonstrates that blood vessel density changes with time following ischemia and is enhanced by the hCNS-SCns grafts. Specifically, hCNS-SCns appear to enhance a natural brain repair mechanism. Therefore, the transplantation of the hCNS-SC grafts (either grown as neurospheres or in an adherent culture) has the clinical potential to restore neovascularization following ischemia.

Other transplanted cells including, for example, human bone marrow cells, human bone marrow-derived cells, and human umbilical cord blood cells, have previously been shown to elicit a similar effect after stroke. (See Shen et al., Neuroscience 137:393 (2006); Chen et al., Circ Res 92:692 (2003); Taguchi et al., J. Clin Invest 114:330 (2004); and Shyu et al., J. Neurosci. 26:3444 (2006)). However, in those studies, the cells were administered much earlier following stroke (day 2) than the hCNS-SCns, which were transplanted in the instant study at day 7. Moreover, in these earlier studies, the effects on neovascularlization were only investigated to two weeks post transplantation. No prior studies have determined whether transplanted human neural stem cells can promote angiogenesis and/or vasculogenesis in the stroke-damaged brain of a host. Likewise, bone marrow, bone marrow-derived and cord blood cells migrate towards the lesion site transiently and disappear from the injury site afterward. Therefore, it is not clear whether these cells can elicit a prolonged effect on neovascularization and/or whether those cells will elicit any effect if administered at a later time after stroke.

In contrast, as demonstrated herein, hCNS-SC do exhibit a prolonged effect on neovascularization. Unlike bone marrow, bone marrow-derived and cord blood cells, hCNS-SC can survive, migrate and reside in the affected area over a long-term period. In cases of spinal cord injury, it has been shown that there is direct link between the survival of hCNS-SC and functional recovery (See Cummings et al. PNAS 102:14069 (2005)). Thus, long-term survival of hCNS-SC presumably contributed to the host neovasculogenesis in this study.

The results presented herein represent the first time that human central nervous system stem cells grown as neurospheres have been shown to enhance neovascularization. A preliminary evaluation of the brain tissue found no difference in the immune reaction of the hCNS-SCns-transplanted and buffer-transplanted brains, which suggests that the observed effect of hCNS-SC on angiogenesis and vasculogenesis is not an indirect result of an hCNS-SC-induced immune response. In addition, no correlation between BVD and the number of monocytes/macrophages was observed, which further suggests that inflammation does not influence the observed neovascularization observed in this study. Furthermore, hCNS-SC-enhanced BVD was found in ischemic nude rats (which are T cell deficient) at 8 weeks post-stroke, implying that T cells are not responsible for this neovascularization effect.

The mechanisms of how hCNS-SC promote host neovascularization following ischemia has not yet been elucidated. One possibility is that the hCNS-SC secrete trophic factors that support neovascularization. Alternatively, a direct cell-cell interaction between the hCNS-SC and host endothelial cells may be required to promote angiogenesis and/or vasculogenesis. In fact, a prior study demonstrated that neurogenesis is associated intimately with vascular recruitment. (See Palmer et al. J Comp Neurol 425:479 (2000)). Thus, hCNS-SC may associate with endothelial cells to promote their proliferation, vascular formation, and sprouting.

Those skilled in the art will recognize that the methods disclosed herein offer potential therapeutic strategies for stroke that can be administered up to 1 week following a stroke. Moreover, based on the results presented herein, it is possible that the transplantation of hCNS-SC could also elicit a similar effect in other models of brain injury. Likewise, other types of stem and progenitor cells could also potentially elicit similar effects on neovascularization following ischemia. Additionally, further investigation regarding how hCNS-SC elicit the observed effects will enable these cells to be engineered to further augment neovascularization, which will potentially lead to better recovery after stroke.

Furthermore, since (a) the hCNS-SCs may elicit an effect through secretion of trophic factors and (b) the hCNS-SCs do not need to differentiate and become mature cells to elicit an effect on neovascularization (the cells are still quite immature at 5 weeks when the effect is observed in the brain), it is possible that hCNS-SCs could augment neovascularization in other tissues in addition to the brain (i.e., despite being of neural origin, the hCNS-SC could also potentiate neovascularization in many different tissues). Thus, hCNS-SCs have the potential to be beneficial in other ischemic diseases (apart from stroke) where neovascularization is important.

### Equivalents

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

We claim:

1. A method of inducing or enhancing neovascularization following ischemia in a patient suffering therefrom, the method comprising transplanting an effective amount of human central nervous system stem cells (hCNS-SC) to one or more lesioned regions of the central nervous system of said patient.

2. The method of claim 1, wherein the hCNS-SC are grown as neurospheres (hCNS-SCns).

3. The method of claim 1, wherein the hCNS-SC are grown in adherent culture.

4. The method of claim 1, wherein the lesioned region of the patient's central nervous system is the ischemic cortex.

5. The method of claim 1, wherein the hCNS-SC are obtained from the host's neural tissue.

6. The method of claim 5, wherein the hCNS-SC are obtained from cerebral cortex tissue, cerebellum tissue, midbrain tissue, brainstem tissue, spinal cord tissue, ventricular tissue, frontal lobe tissue, conus medularis tissue, hypothalamus tissue, or a combination thereof.

7. The method of claim 1, wherein the hCNS-SC are obtained from embryonic, fetal, juvenile, or adult human neural tissue.

8. The method of claim 7, wherein the hCNS-SC are obtained from cerebral cortex tissue, cerebellum tissue, midbrain tissue, brainstem tissue, spinal cord tissue, ventricular tissue, frontal lobe tissue, conus medularis tissue, hypothalamus tissue, or a combination thereof.

9. The method of claim 1, wherein at least 1×10⁵ cells are injected at each lesioned region.

10. The method of claim 1, wherein transplantation of said hCNS-SC results in increased angiogenesis, increased vasculogenesis, or both within the cortical ischemic penumbra.

11. A method for inducing the repair of ischemic tissue in a patient, the method comprising transplanting an effective amount of human central nervous system stem cells (hCNS-SC) at one or more loci within the central nervous system of the patient, wherein said hCNS-SC are able to increase angiogenesis, vasculogenesis, or both within said ischemic tissue.

12. The method of claim 11, wherein hCNS-SC are grown as neurospheres (hCNS-SCns).

13. The method of claim 11, wherein the hCNS-SC are grown in adherent culture.

14. The method of claim 11, wherein the hCNS-SC are obtained from the host's neural tissue.

15. The method of claim 14, wherein the hCNS-SC are obtained from cerebral cortex tissue, cerebellum tissue, midbrain tissue, brainstem tissue, spinal cord tissue, ventricular tissue, frontal lobe tissue, conus medularis tissue, hypothalamus tissue, or a combination thereof.

16. The method of claim 11, wherein the hCNS-SC are obtained from embryonic, fetal, juvenile, or adult human neural tissue.

17. The method of claim 16, wherein the hCNS-SC are obtained from cerebral cortex tissue, cerebellum tissue, midbrain tissue, brainstem tissue, spinal cord tissue, ventricular tissue, frontal lobe tissue, conus medularis tissue, hypothalamus tissue, or a combination thereof.

18. The method of claim 11, wherein at least 1×10⁵ cells are injected per loci.

19. A method for treating a stroke in a patient, the method comprising injecting at least 1×10⁵ human central nervous system stem cells (hCNS-SC) at one or moreloci within the ischemic cortex of the central nervous system of the patient suffering therefrom, wherein said hCNS-SC are able to increase neovascularization in the ischemic cortex, thereby aiding neurological recovery in said patient.

20. The method of claim 19, wherein the hCNS-SC are grown as neurospheres (hCNS-SCns).

21. The method of claim 19, wherein the hCNS-SC are grown in adherent culture.

22. The method of claim 19, wherein the hCNS-SC are obtained from the host's neural tissue.

23. The method of claim 16, wherein the hCNS-SC are obtained from cerebral cortex tissue, cerebellum tissue, midbrain tissue, brainstem tissue, spinal cord tissue, ventricular tissue, frontal lobe tissue, conus medularis tissue, hypothalamus tissue, or a combination thereof.

24. The method of claim 15, wherein the hCNS-SC are obtained from embryonic, fetal, juvenile, or adult human neural tissue.

25. The method of claim 18, wherein the hCNS-SC are obtained from cerebral cortex tissue, cerebellum tissue, midbrain tissue, brainstem tissue, spinal cord tissue, ventricular tissue, frontal lobe tissue, conus medularis tissue, hypothalamus tissue, or a combination thereof.
